(19) United States
(12) Patent Application Publication
(10) Pub. No.: US 2023/0349860 A1
(43) Pub. Date: Nov. 2, 2023

(54) MAGNETIC SIGNAL DETECTION CHIP, DETECTION CARD AND NUCLEIC ACID DETECTION DEVICE

(71) Applicant: TDK CORPORATION, Tokyo (JP)

(72) Inventors: Jacky HO, Nancheng Dongguan (CN); Yu Qing SHI, Nancheng Dongguan (CN)

(73) Assignee: TDK CORPORATION, Tokyo (JP)

(21) Appl. No.: 17/914,400

(22) PCT Filed: Mar. 30, 2021
(86) PCT No.: PCT/IB2021/052600

§ 371 Date: Sep. 26, 2022

(30) Foreign Application Priority Data
Mar. 31, 2020 (CN) 202010245205.1

(51) Int. Cl. G01N 27/74; G01N 33/543; G01N 33/53

(52) U.S. Cl. CPC G01N 27/745; G01N 33/54326; G01N 33/5308

(57) ABSTRACT

Provided is a magnetic signal detection chip in which taking the centerline of a sensing region of the magnetic signal detection chip extending along the flow direction of the chemical fluid as a reference, each of sensing units on one side is connected to a corresponding output pad on the one side via lead, while each of sensing units on the other side is connected to a corresponding output pad on the other side via lead. In this way, the size of the magnetic signal detection chip could be reduced and the lead resistance could be reduced. Further provided is a detection card including the magnetic signal detection chip and further provided is a nucleic acid detection device including the detection card.

### TECHNICAL FIELD

The present disclosure relates to a technique for detecting the composition of chemical substance by means of magnetic signals, in particular a magnetic signal detection chip, a detection card including the magnetic signal detection chip, and a nucleic acid detection device including the detection card.

### BACKGROUND

In the prior art, a detection card including a magnetic signal detection chip is used to quantitatively detect the composition of chemical substances by means of magnetic signals. For example, in a Chinese invention patent application with the publication number CN109917139A titled with “GMR chip and magnetic sensitive immunodetection card including the same” has disclosed a GMR chip and a magnetic sensitive immunodetection card for protein detection. However, in the prior art represented by the above-mentioned Chinese invention patent application, the size of the GMR chip is very large and the lead length between the sensing unit and the output pad is very long, resulting in higher lead resistance. These defects bring adverse effects on the miniaturization of the detection card, the cost of the detection card, and the detection result of the detection card.

### SUMMARY

The present disclosure was made in view of the above-mentioned defects of the prior art. An object of the present disclosure is to provide a novelty magnetic signal detection chip, which is smaller in size and lower in lead resistance relative to the magnetic signal detection chip in the prior art. Another object of the present disclosure is to provide a detection card including the magnetic signal detection chip and a nucleic acid detection device including the detection card.

In order to obtain the above objects, the present disclosure has the following technical solutions.

This disclosure provides a magnetic signal detection chip, comprising:
a plurality of sensing units, which are arranged inside a sensing region of the magnetic signal detection chip;
a plurality of output pads, which are arranged outside the sensing region and are located on both sides of the sensing region, wherein taking a first centerline of the sensing region extending along a flow direction of chemical fluid inside the sensing region as a reference, each of the sensing units on one side is connected to a corresponding output pad on the one side via lead, while each of the sensing units on the other side is connected to a corresponding output pad on the other side via lead; and
a plurality of the leads, each of the leads is connected to the sensing unit and the output pad.

Preferably, on both sides of the sensing region, the output pads are arranged in one row respectively, which extends along the flow direction.

More preferably, the number of the output pads on the one side is equal with the number of the output pads on the other side.

More preferably, in the flow direction, the region where the output pads are arranged does not exceed the region where the sensing units are arranged.

More preferably, the plurality of sensing units are divided into multiple groups, and multiple groups of the sensing units are disposed in a symmetrical manner with respect to a second centerline of the sensing region, wherein the second centerline extends along a direction orthogonal to the flow direction.

More preferably, the plurality of sensing units are arranged in multiple rows, which extend along the flow direction of the chemical fluid, wherein the sensing units in one row are staggered from the sensing units in an adjacent row.

More preferably, the magnetic signal detection chip further comprises common ground pads, and wherein the ground pads are arranged outside the sensing region and connected to the plurality of sensing units via leads.

More preferably, on both sides of the sensing region, the output pads are arranged in one row respectively, which extends along the flow direction of the chemical fluid, wherein the ground pads are arranged in the row of the output pads.

This disclosure further provides a detection card, comprising the magnetic signal detection chip according to any one of the above technical solutions.

Preferably, the detection card further comprises a printed. circuit board, the magnetic signal detection chip is provided on the printed circuit board, and the printed circuit board comprises a plurality of bond pads and a plurality of wires, wherein the output pad on one side of the magnetic signal detection chip is connected to the bond pad on the one side via the wire, while the output pad on the other side of the magnetic signal detection chip is connected to the bond pad on the other side via the wire.

This disclosure further provides a nucleic acid detection device, comprising the detection card according to any one of the above technical solutions.

Upon the above technical solutions, the present disclosure provides a magnetic signal detection chip, a detection card including the magnetic signal detection chip, and a nucleic acid detection device including the detection card. In the magnetic signal detection chip, a plurality of sensing units are arranged inside a sensing region of the magnetic signal detection chip, and a plurality of output pads are arranged outside the sensing region and are respectively located on both sides of the sensing region. Further, taking the centerline of the sensing region extending along the flow direction of the chemical fluid as the reference, each of the sensing units on one side is connected to a corresponding output pad on the one side via lead, while each of the sensing units on the other side is to connected to a corresponding output pad on the other side via lead. In this way, in the technical solution of the disclosure, on the one hand, the output pads are arranged on both sides of the sensing region, rather than on one side as in the prior art; on the other hand, each of the sensing unit is connected is the output pad via lead which is close to the sensing unit, instead of connecting the sensing unit to an output pad far away from sensing unit as in the prior art. Therefore, compared with the prior art, the size of the magnetic signal detection chip could be reduced and the lead resistance could be reduced, so that the magnetic signal detection chip and the detection card including such chip could be miniaturized, and the cost of the magnetic signal detection chip and the detection card could be reduced, the adverse effect of large lead resistance on the detection results of the magnetic signal detection chip and the detection card is avoided.

### BRIEF INTRODUCTION OF THE DRAWINGS

FIG. 1A is a schematic view showing the structure of the magnetic signal detection chip according to the first embodiment of the present disclosure, in which the hollow double-headed arrows indicates the flow direction of the chemical fluid flowing through the sensing region of the magnetic signal detection chip; FIG. 1B is a schematic view showing the structure of the portion, where the magnetic signal detection chip shown in FIG. 1A is connected to the bond pads on the printed circuit board via wires.

FIG. 2 is a schematic view showing the structure of a magnetic signal detection chip according to the second embodiment of the present disclosure.

FIG. 3 is a schematic view showing the cross-section structure of the detection card including the magnetic signal detection chip according to the first embodiment.

FIG. 4 is a schematic view showing a nucleic acid detection device including the above detection card.

### LIST OF REFERENCE SIGNS

1 magnetic signal detection chip
11 sensing unit
12 output pad
13 ground pad
14 lead
21 bond pad
22 wire
23 printed circuit board
3 silica gel
C detection card
F flow direction
X first centerline
Y second centerline
R sensing region

### DETAILED EMBODIMENTS

Exemplary embodiments of the present disclosure will be described below with reference to the drawings. It should be understood that these specific descriptions are only intended to teach those skilled in the art how to practice the present disclosure, and are not intended to be exhaustive of all possible ways of carrying out the present disclosure or to limit the scope of the present disclosure.

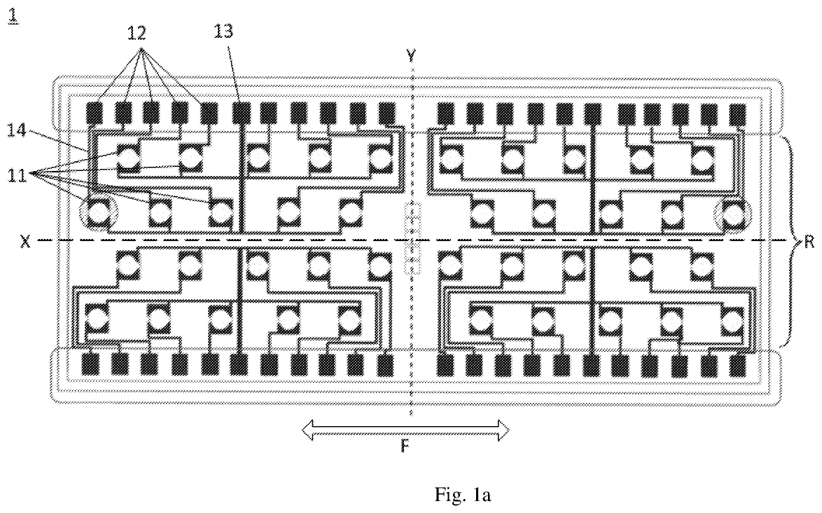

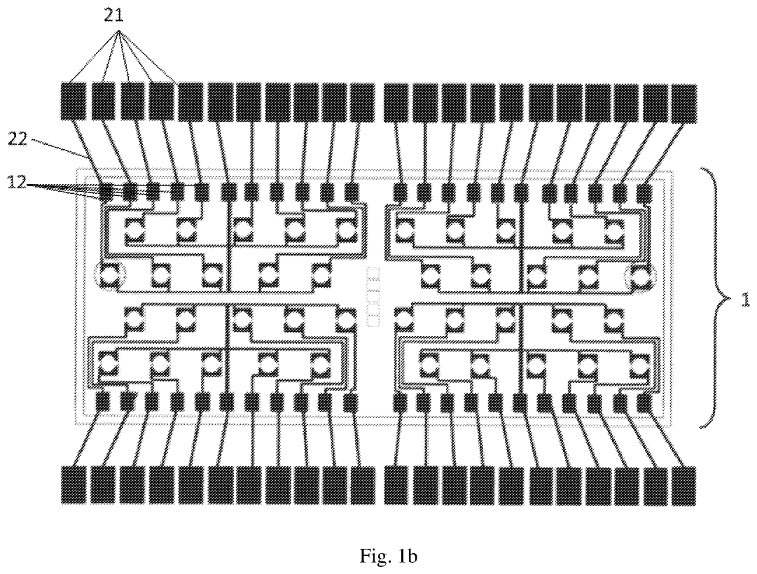

It should be noted that in the present disclosure, “both sides” refer to both sides of the first centerline X of the magnetic signal detection chip, while “one side” refers to the upper side in FIGS. 1A and 1B and “the other side” refers to the lower side in FIGS. 1A and 1B.

The structure of the magnetic signal detection chip according to the first embodiment of the present disclosure will be described below with reference to the drawings.

### Structure of the Magnetic Signal Detection Chip According to the First Embodiment of the Present Disclosure

As shown in FIGS. 1A and 1B, the magnetic signal detection chip 1 according to the first embodiment of the present disclosure includes a plurality of sensing units 11, a plurality of output pads 12, a plurality of ground pads 13 and a plurality of leads 14, which are provided on the matrix of the magnetic signal detection chip 1.

Specifically, in this embodiment, a plurality of sensing units 11 are arranged inside the sensing region R of the magnetic signal detection chip 1, and are used to sense the magnetic beads in the chemical fluid flowing through the sensing region R, so as to quantitatively analyze the precise content of the components to be detected in the chemical fluid. The sensing region R may be either a flow path in which the chemical fluid flows from the left side in FIG. 1A toward the right side in FIG. 1A or a flow path in which the chemical fluid flows from the right side in FIG. 1A toward the left side in FIG. 1A, wherein the flow path may be defined by the counter structure of the detection card including the magnetic signal detection chip 1. In FIG. 1A, the range of the sensing region R on both sides is indicated by angle brackets.

In this embodiment, the sensing units 11 are arranged in four rows, which extend along the flow direction F of the chemical fluid, wherein the sensing units 11 in one row are staggered from the sensing units 11 in an adjacent row in the flow direction F, so as to facilitate the arrangement of the leads 14 from each of the sensing units 11. On the other perspective, the sensing units 11 are divided into four groups, and the number of the sensing units 11 in the each group is equal. The four groups of sensing units 11 are arranged in a symmetrical manner with respect to the second centerline Y of the sensing region R extending in a direction orthogonal to the flow direction F. The regular layout of the sensing units 11 built in this way is beneficial to the miniaturization of the magnetic signal detection chip 1.

In this embodiment, the number of the output pads 12 is equal to the number of the sensing units 11, and the output pads 12 are connected with the sensing units 11 in a one-to-one mapping through the leads 14, so that the signal generated by the sensing unit: 11 is outputted outside the magnetic signal detection chip 1. The plurality of output pads 12 are arranged outside the sensing region R, so that the plurality of output pads 12 will not be in contact with the chemical fluid, in the present disclosure, different from the prior art, the plurality of output pads 12 are located on both sides of the sensing region R, wherein taking the first centerline X of the sensing region R extending along the flow direction F of the chemical fluid as a reference, the sensing units 11 on one side is connected to a corresponding output pad 12 on the one side via lead 14, while each of the sensing units 11 on the other side is connected with a corresponding output pad 12 on the other side via lead 14. In this way, not only the size of the magnetic signal detection chip 1 could be reduced, but also the length of the leads 14 could he reduced, when each sensing unit 11 is connected to the output pad 12 close to the sensing unit 11 via the lead 14, so as to reduce the resistance of the leads 14 compared with the prior art.

Further, on both sides of the sensing region R, the output pads 12 are respectively arranged in a row, which extends along the flow direction F. In this way, compared with the output pads 12 arranged in multiple rows on one side in the prior art, the size of the magnetic signal detection chip 1 could be further reduced and the length of the leads 14 could be further reduced. Further, the number of the output pads 12 on one side of the sensing region R is equal to that on the other side. In this way, the problems in routing caused by too dense arrangement of the output pads 12 on one side can be avoided. Further, the region where the output pads 12 are arranged does not exceed the region where the sensing units 11 are arranged. In this way, it could be ensured that the size of the magnetic signal detection chip 1 in the flow direction F will not become larger due to the excessive extension of the arrangement region of the output pads 12.

Further, corresponding to the division of the sensing units 11 into four groups, in this embodiment, the output pads 12 are also divided into four groups. Each group of output pads 12 is respectively connected to one group of sensing units 11, and each group of output pads 12 is arranged at a position directly close to the corresponding group of sensing units 11, so that the length of the leads 14 between the output pads 12 and the sensing units 11 could be further reduced.

In this embodiment, each ground pad 13 is provided corresponding to a plurality of sensing units 11 and is shared by the plurality of sensing units 11. The ground pad 13 is used for grounding rather than outputting signals of the sensing unit 11 to outside. Similar to the output pads 12 described above, the ground pads 13 are arranged outside the sensing region R and each ground pad 13 is connected to a plurality of sensing units 11 through leads 14. Corresponding to the division of the sensing unit 11 into four groups, in this embodiment, four ground pads 13 are provided. Each ground pad 13 is connected to only one group of sensing units 11 via leads 14. It means the ground pad 13 is a common pad to serve a group of sensing units 11. In addition, the ground pad 13 corresponding to a group of sensing units 11 and the output pads 12 corresponding to the group of sensing units 11 are arranged in a row, so that the size of the magnetic signal detection chip would not become larger due to the arrangement of the ground pad 13.

In this embodiment, one end of each lead 14 is connected to a sensing unit 11, and the other end of each lead 14 is connected to a output pad 12 or a ground pad 13. Corresponding to the division of the sensing unit 11 into four groups, in this embodiment, the layout of the leads 14 is also divided into four parts, and the leads 14 should be laid out as short as possible without affecting the normal operation of other components.

Referring to FIGS. 1A and 1B, in each group of sensing units 11, for the row of sensing units 11 far away from the first centerline X (i.e., the row of sensing units 11 on the outside), the leads 14 substantially do not exceed the region of the sensing units 11 in the flow direction F, while the output pads 12 corresponding to the row of the sensing unit 11 on the outside are located in the middle region of the group in the flow direction F.

For the row of sensing units 11 close to the first centerline X (i.e., the row of sensing units 11 on the inner side), in the flow direction F, the leads 14 pass round two sides of the sensing units 11 on the outside, and the output pads 12 corresponding to the row of sensing units 11 on the inside are located in the regions on two sides in the flow direction F.

In this way, the sensing units 11 could be arranged compacter, and the length of the leads 14 could be reduced.

The leads 14 connecting the plurality of sensing units 11 with the ground pad 13 are not connected to the ground pad 13 independently of each other, but the leads drawn from the plurality of sensing units 11 are first gathered together and then connected to the ground pad 13 by a single lead separately, which simplifies the layout of the leads connected to the ground pad 13 and reduces the routing complexity and the length of the lead 14.

The structure of the magnetic signal detection chip I according to the first embodiment of the present disclosure has been described above, and the structure of the detection card according to the present disclosure including the magnetic signal detection chip 1 will be described below.

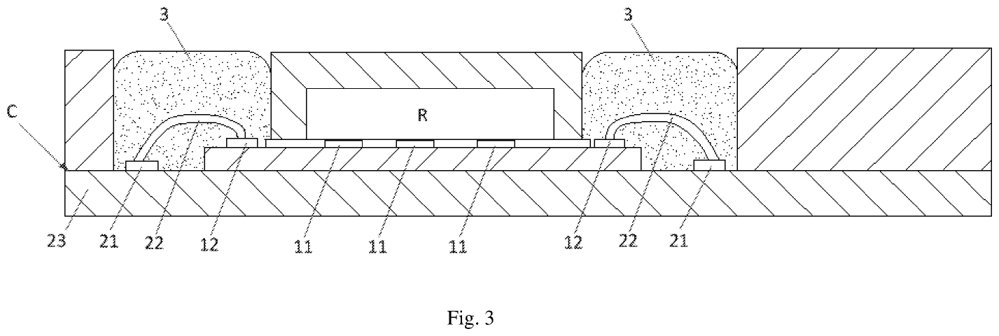

As shown in FIG. 3, the detection card C according to the present disclosure includes not only the above-mentioned magnetic signal detection chip 1, but also a printed circuit board 23, wherein the magnetic signal detection chip 1 is provided on the printed circuit board 23. As shown in FIGS. 1B and 3, the printed circuit board 23 includes a plurality of bond pads 21 and a plurality of wires 22, and the plurality of bond pads 21 are located on both sides of the magnetic signal detection chip 1. Further, the output pads 12 and the ground pads 13 on one side of the magnetic signal detection chip 1 are connected to the bond pads 21 on the same side via wires 22, respectively, while the output pads 12 and the ground pads 13 on the other side of the magnetic signal detection chip 1 are connected to the bond pads 21 on the same side via wires 22, respectively. In this way, similar to the principle that the sensing units 11 of the magnetic signal detection chip 1 are connected to the output pads 12 and the ground pads 13 on the same side, the length of the wires 22 in the above structure could be significantly reduced. Additionally, the bond pads 21, the wires 22 and the output pads 12 (as well as the ground pads 13 which are not shown in FIG. 3) are embedded into silica gel 3, to be protected from being unexpectedly touched or contamination. Furthermore, the sensing region R is limited by a cover located above the detection chip.

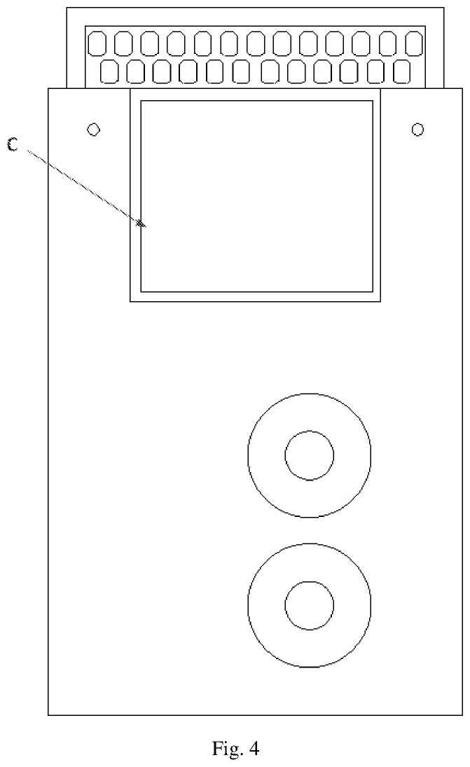

In addition, as shown in FIG. 4, the present disclosure also provides a nucleic acid detection device including the above detection card C. The nucleic acid detection device is used for detecting the content of nucleic acid in a chemical fluid by the detection card C. The nucleic acid detection device includes a housing for example made of resin and a detection card C partially accommodated in the housing, wherein a part of the printed circuit board 23 of the detection card C including the bond pads 21 may be exposed from the housing.

The structure of the magnetic signal detection chip 1 according to the first embodiment of the present disclosure and the structure of the detection card including the magnetic signal detection chip I have been described above, and the structure of the magnetic signal detection chip 1 according to the second embodiment of the present disclosure will be described below,

### Structure of the Magnetic Signal Detection Chip 1 According to the Second Embodiment of the Present Disclosure

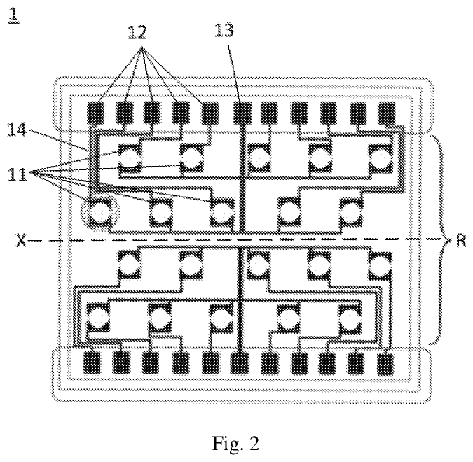

As shown in FIG. 2, the basic structure of the magnetic signal detection chip 1 according to the second embodiment of the present disclosure is similar to the basic structure of the magnetic signal detection chip 1 according to the first embodiment of the present disclosure, and the differences between the two embodiments will be explained below

In this embodiment, corresponding to the reduction in the types of components required to be detected in the chemical fluid, the number of sensing units 11 of the magnetic signal detection chip 1 is reduced to half compared with the first embodiment. In this way, the sensing unit 11 in this embodiment is only divided into two groups, one group is located on one side of the first centerline X, and the other group is located on the other side of the first centerline X. Although in this embodiment the number of sensing units 11 of the magnetic signal detection chip 1 is reduced, technical effect of reducing the size of the magnetic signal detection chip 1 and reducing the length of the leads 14 is obtained in this embodiment of the present disclosure compared with magnetic signal detection chip in the prior art having the same number of sensing units.

In summary, the present disclosure provides a novelty magnetic signal detection chip 1, a detection card including the magnetic signal detection chip 1, and a nucleic acid detection device including the detection card, which are however not limited to those examples listed in the above detailed embodiments. Some supplementary explanations are provided below.
i. Although not described clearly in the above detailed embodiments, it could be understood that the magnetic signal detection chip 1 according to the present disclosure is a magnetic induction element, wherein Giant Magneto Resistance chip (GMR chip) or a tunnel Magneto Resistance chip (TMR chip) could be used, and Hall chip could also be used. Preferably, the magnetic signal detection chip 1 according to the present disclosure is a Giant Magneto Resistance chip.
ii. Although the magnetic signal detection chip 1 with a specific number of sensing units 11 is described in the above detailed embodiments, the present disclosure is not limited to this. The number of sensing units 11 of the magnetic signal detection chip 1 could be determined depending on the number of components of the chemical fluid to be detected.
iii. Although it is described in the above detailed embodiments that the sensing units 11 of the magnetic signal detection chip 1 are divided into multiple groups, the present disclosure is not limited to this. As long as the sensing units 11 are connected to the output pad 12 on the same side, it is not necessary to to divide the sensing units 11 into multiple groups as described in the above detailed embodiments.
iv. Although not described in the above first embodiment, it could be understood that in this embodiment, the leads 14 is also routed in a symmetrical manner with respect to the second centerline Y. Moreover, for a group of sensing units 11, the leads 14 of the sensing unit 11 on the left side are routed from the left side of the group of sensing units 11, while the leads 14 of the sensing unit 11 on the right side are routed from the right side of the group of sensing units 11. Such layout of the leads 14 could prevent all the leads 14 from being routed on the left together or right side together, which results in an increase in the size of the magnetic signal detection chip 1.

1. A magnetic signal detection chip, comprising:
a plurality of sensing units, which are arranged inside a sensing region of the magnetic signal detection chip;
a plurality of output pads, which are arranged outside the sensing region and are located on both sides of the sensing region, wherein taking a first centerline of the sensing region extending along a flow direction of chemical fluid inside the sensing region as a reference, each of the sensing units on one side is connected to a corresponding output pad on the one side via lead, while each of the sensing units on the other side is connected to a corresponding output pad on the other side via lead; and
a plurality of the leads, each of the leads is connected to the sensing unit and the output pad.

2. The magnetic signal detection chip according to claim 1, wherein on both sides of the sensing region, the output pads are arranged in one row respectively, which extends along the flow direction.

3. The magnetic signal detection chip according to claim 1, wherein the number of the output pads on the one side is equal with the number of the output pads 12 on the other side.

4. The magnetic signal detection chip according to claim 1, wherein in the flow direction, the region where the output pads are arranged does not exceed the region where the sensing units are arranged.

5. The magnetic signal detection chip according to claim 1, wherein the plurality of sensing units are divided into multiple groups, and multiple groups of the sensing units are disposed in a symmetrical manner with respect to a second centerline of the sensing region, wherein the second centerline extends along a direction orthogonal to the flow direction.

6. The magnetic signal detection chip according to claim 1, wherein the plurality of sensing units are arranged in multiple rows, which extend along the flow direction of the chemical fluid, wherein the sensing units in one row are staggered from the sensing units in an adjacent row.

7. The magnetic signal detection chip according to claim 1, wherein the magnetic signal detection chip further comprises common ground pads, and wherein the ground pads are arranged outside the sensing region and connected to the plurality of sensing units via leads.

8. The magnetic signal detection chip according to claim 7, wherein on both sides of the sensing region, the output pads are arranged in one row respectively, which extends along the flow direction of the chemical fluid, wherein the ground pads are arranged in the row of the output pads.

9. A detection card, comprising a magnetic signal detection chip comprising:
a plurality of sensing units, which are arranged inside a sensing region of the magnetic signal detection chip;
a plurality of output pads, which are arranged outside the sensing region and are located on both sides of the sensing region, wherein taking a first centerline of the sensing region extending along a flow direction of chemical fluid inside the sensing region as a reference, each of the sensing units on one side is connected to a corresponding output pad on the one side via lead, while each of the sensing units on the other side is connected to a corresponding output pad on the other side via lead; and
a plurality of the leads, each of the leads is connected to the sensing unit and the output pad.

10. The detection card according to claim 9, wherein the detection card further comprises a printed circuit board, the magnetic signal detection chip is provided on the printed circuit board, and the printed circuit board comprises a plurality of bond pads and a plurality of wires, wherein the output pad on one side of the magnetic signal detection chip is connected to the bond pad on the one side via the wire, while the output pad on the other side of the magnetic signal detection chip is connected to the bond pad on the other side via the wire.

11. A nucleic acid detection device, comprising a detection card, which comprising a magnetic signal detection chip comprising:
a plurality of sensing units, which are arranged inside a sensing region of the magnetic signal detection chip;
a plurality of output pads, which are arranged outside the sensing region and are located on both sides of the sensing region, wherein taking a first centerline of the sensing region extending along a flow direction of chemical fluid inside the sensing region as a reference, each of the sensing units on one side is connected to a corresponding output pad on the one side via lead, while each of the sensing units on the other side is connected to a corresponding output pad on the other side via lead; and
a plurality of the leads, each of the leads is connected to the sensing unit and the output pad.
